(19) United States
(12) Patent Application Publication
(10) Pub. No.: US 2024/0229051 A9
(43) Pub. Date: Jul. 11, 2024

(54) CHLOROPLAST CYTOSINE BASE EDITORS AND MITOCHONDRIA CYTOSINE BASE EDITORS IN PLANTS

(71) Applicants: The Curators of the University of Missouri, Columbia, MO (US); Donald Danforth Plant Science Center (DDPSC), St. Louis, MO (US)

(72) Inventors: Bing Yang, Columbia, MO (US); Sinian Char, Columbia, MO (US); Riqing LI, Columbia, MO (US)

(21) Appl. No.: 18/546,837

(22) PCT Filed: Feb. 17, 2022
(86) PCT No.: PCT/US2022/016792

§ 371 Date: Aug. 17, 2023

Related U.S. Application Data
(60) Provisional application No. 63/150,123, filed on Feb. 17, 2021.

(51) Int. Cl. C12N 15/82; C07K 14/415; C12N 9/22; C12N 9/78

(52) U.S. Cl. CPC C12N 15/8214; C07K 14/415; C12N 9/22; C12N 9/78; C12N 15/8213; C07K 2319/08; C12Y 305/04005

(57) ABSTRACT

The present disclosure is generally directed to gene editing in plant chloroplast and plant mitochondrial double-stranded DNA. Disclosed herein are cytosine base editors tailored for chloroplast and mitochondrial genomes in plants using plant-specific chloroplast and mitochondrial targeting peptides, a TALE, and a DNA deaminase. The systems of the present disclosure include DNA vectors and protocols to use them for gene editing in plants.

### CROSS-REFERENCE TO RELATED APPLICATIONS

This application claims priority under 35 U.S.C. 119(e) to United States Provisional Patent Application Ser. No. 63/150,123, filed on Feb. 17, 2021, the disclosure of which is hereby incorporated by reference in its entirety.

### STATEMENT OF GOVERNMENT SUPPORT

This invention was made with government support under grant award number IOS 1936492 awarded by the National Science Foundation. The government has certain rights in the invention.

### INCORPORATION OF SEQUENCE LISTING

A paper copy of the Sequence Listing and a computer readable form of the Sequence Listing containing the file named “21UMC035_PCT_ST25.txt”, which is 55649 bytes in size as measured in MICROSOFT WINDOWS EXPLORER®, are provided herein and are herein incorporated by reference. This Sequence Listing consists of SEQ ID NOs:1-43.

### BACKGROUND OF THE DISCLOSURE

The present disclosure relates generally to compositions and methods for gene editing in plants. In particular, the present disclosure relates to compositions and methods for editing plant chloroplast and plant mitochondrial nucleic acids.

Genome editing holds promise in basic and applied research in life science, medicine and agriculture. It depends on the enzymatic reagents to change genomic DNA, leading to genetic changes in genomes (e.g., nuclear, chloroplast, mitochondria) of interest in a stable and transmittable way (used for animal or crop breeding) or no transmittable way such as in somatic cells (used for gene therapy). The editing reagents include engineered zinc finger nucleases (ZFNs), transcription activator-like effector nucleases (TALENs), and clustered regularly interspaced short palindromic repeats (CRISPR) and CRISPR associated proteins (Cas) (CRISPR/Cas). These reagents either introduce doubled stranded DNA breaks (DSBs) or chemical alteration of DNA bases at the user-chosen genomic sites. The DNA repair to the DSBs and chemical changes by the repairing process leads to the desired DNA changes, including those that encode improved agronomic traits in crop plants.

CRISPR based genome editing is the most widely used technology to edit the nuclear genomes but not feasible to edit the organelle genomes due to difficulty in delivery of CRISPR guide RNA into the organelles of eukaryotic organisms. To overcome such limitation, TALENs have been shown to be targeted into mitochondria and perform gene editing in mitochondrial genomes by fusing the mitochondrial transition peptide to the TALENs (mtTALENs) in rice (Kazama et al. 2019). The limitation of mtTALENs is the inefficient repair of DSBs and thus very low gene editing efficiency in mitochondria or organelles in general. Recently, Mok et al. (Nature 2020, 583(7817):631-637) demonstrated a technology for mitochondrial gene editing in human cells by. In their work, Mok and colleagues fused a part of a bacterium-derived cytosine deaminase domain, namely DddA, to one of paired TALE DNA binding domains and fused another split-half to another TALE. Both fusion proteins are each further fused with a mitochondrial transition peptide and an uracil glycosylase inhibitor (UGI). The split-halves need to come together to deaminate cytidines of the double stranded DNA. Expression of the fusion proteins in HEK293T cells resulted in DddA-derived cytosine base editors (DdCBEs) that catalyze C·G to T·A conversions in human mtDNA with high target specificity.

TALEs of bacterial origin recognize DNA sequences of target sites following a TALE DNA recognition code, i.e., one modular repeat of 34 amino acids corresponds to one nucleotide and four predominant repeats recognize four nucleotides respectively (Boch et al. 2009 Breaking the code of DNA binding specificity of TAL-type III effectors. Science 326: 1509-1512; Moscou et al. 2009 A simple cipher governs DNA recognition by TAL effectors. Science 326: 1501). Specifically, two amino acids at the position 12 and 13, so-called repeat variable di-residues (RVDs), of the 34 amino acids determine the specificity of nucleotide recognition, e.g., NI, HD, NG and NN corresponding to A, C, T, G for DNA binding. TALE DNA binding domains can be modularly assembled based on the TALE DNA recognition code and the preselected genomic sequences (Li et al. 2011 Modular assembled designer TAL effector nucleases for targeted gene knockout and gene replacement in eukaryotes. Nucleic Acids Research 39: 6315-6425). When two paired TALE DNA binding domains are located at the nearby sites on the opposite strands (sense and antisense strands) in an appropriate distance (spacer) that the enzymatic domains (e.g., endonuclease domains—for TALENs, DddA deaminase domain—for TALCDA)) can function on DNA, e.g., leading to DSBs or cytidine deamination.

Disclosed herein are cytosine base editors tailored for chloroplast and mitochondrial genomes in plants. The cytosine base editors use plant-specific transition signal peptides for chloroplast and mitochondria targeting, TALE, deaminase and uracil glycosylase inhibitor. The systems of the present disclosure include a serial of DNA vectors and protocols to use them.

### BRIEF DESCRIPTION OF THE DISCLOSURE

The present disclosure is generally directed to compositions and methods for performing gene editing in plant chloroplasts and plant mitochondria.

In one aspect, the present disclosure is directed to a recombinant fusion protein comprising a targeting peptide selected from the group consisting of a plant chloroplast targeting peptide and a plant mitochondrial targeting peptide, a TALE array protein, and a deaminase.

In one aspect, the present disclosure is directed to a recombinant fusion protein comprising a targeting peptide selected from the group consisting of a plant chloroplast targeting peptide and a plant mitochondrial targeting peptide, a TALE array protein, a deaminase, and at least one uracil glycosylase inhibitor.

In one aspect, the present disclosure is directed to a nucleic acid encoding a recombinant fusion protein comprising a targeting peptide selected from the group consisting of a plant chloroplast targeting peptide and a plant mitochondrial targeting peptide, a TALE array protein, and a deaminase.

In one aspect, the present disclosure is directed to a nucleic acid encoding a recombinant fusion protein comprising a targeting peptide selected from the group consisting of a plant chloroplast targeting peptide and a plant mitochondrial targeting peptide, a TALE array protein, a deaminase, and at least one uracil glycosylase inhibitor.

In one aspect, the present disclosure is directed to a vector comprising a nucleic acid encoding a recombinant fusion protein comprising a targeting peptide selected from the group consisting of a plant chloroplast targeting peptide and a plant mitochondrial targeting peptide, a TALE array protein, and a deaminase.

In one aspect, the present disclosure is directed to a vector comprising a nucleic acid encoding a recombinant fusion protein comprising a targeting peptide selected from the group consisting of a plant chloroplast targeting peptide and a plant mitochondrial targeting peptide, a TALE array protein, a deaminase, and at least one uracil glycosylase inhibitor.

In one aspect, the present disclosure is directed to a method of editing plant chloroplast DNA, the method comprising: providing a nucleic acid encoding a recombinant fusion protein comprising a plant chloroplast targeting peptide, a TALE array protein, and a deaminase.

In one aspect, the present disclosure is directed to a method of editing plant chloroplast DNA, the method comprising: providing a nucleic acid encoding a recombinant fusion protein comprising a plant chloroplast targeting peptide, a TALE array protein, a deaminase, and at least one uracil glycosylase inhibitor.

In one aspect, the present disclosure is directed to a method of editing plant mitochondrial DNA, the method comprising: providing a nucleic acid encoding a recombinant fusion protein comprising a plant mitochondrial targeting peptide, a TALE array protein, and a deaminase.

In one aspect, the present disclosure is directed to a method of editing plant mitochondrial DNA, the method comprising: providing a nucleic acid encoding a recombinant fusion protein comprising a plant mitochondrial targeting peptide, a TALE array protein, a deaminase, and at least one uracil glycosylase inhibitor.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a schematic illustrating a nucleic acid encoding a cytosine base editor and a selection marker (e.g., Hyg, Bar, or GFP) which when expressed provides two inactive recombinant fusion proteins, one that includes the N-terminal domain of a deaminase (e.g., “TALCDA-L”) and one that includes the C-terminal domain of the deaminase (e.g., “TALCDA-R”). Upon targeting to a plant chloroplast (“cp”) or plant mitochondria (“mt”), the TALCDA-L and TALCDA-R bind neighboring sites in a target DNA to reconstitute an active deaminase that can mediate gene editing at the target DNA site.

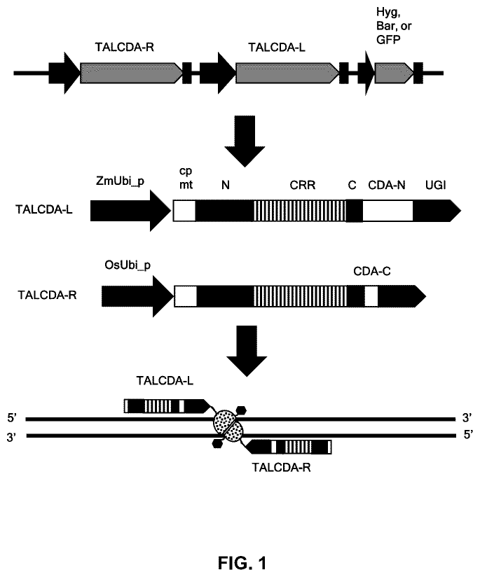

FIG. 2 is a schematic illustrating the structure of PsbA targeted by a cytosine base editor and a representative of Sanger sequencing chromatogram indicating C·G to T·A conversion in the PsbA gene in transgenic rice plants. Rice plants were generated by introducing DNA constructs expressing TALCDAs targeting the PsbA gene. Genomic DNA samples were extracted from leaves of individual transgenic plants. PCR-amplicons from the targeted region were subjected to Sanger sequencing. Arrows indicate the nucleotide conversions. The spacer region is shaded.

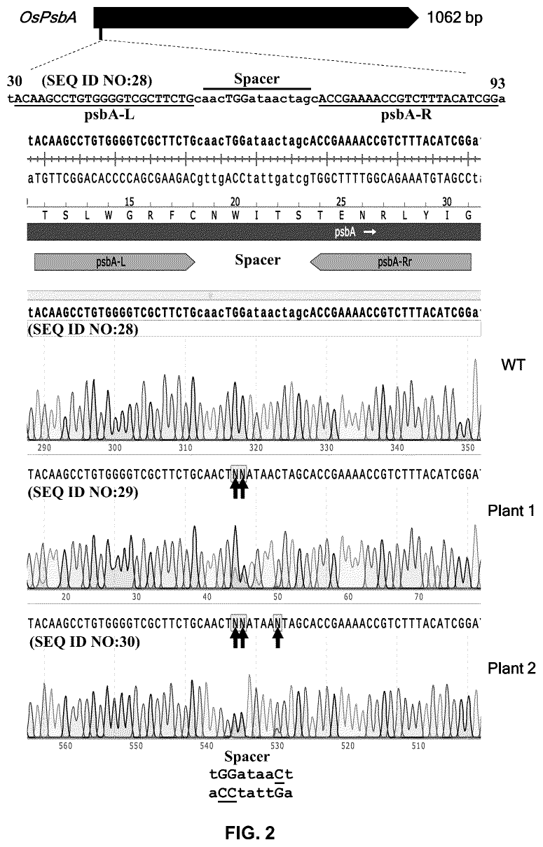

FIG. 3 is a schematic illustrating the structure of PsaA targeted by a cytosine base editor and a representative of Sanger sequencing chromatogram indicating C·G to T·A conversion in PsaA in wheat transgenic plants. Wheat transgenic plants were generated by introducing DNA constructs expressing TALCDAs targeting the PsaA gene and subjected to DNA extraction. PCR-amplicons from the targeted region were subjected to Sanger sequencing. Arrows indicate the nucleotide conversions.

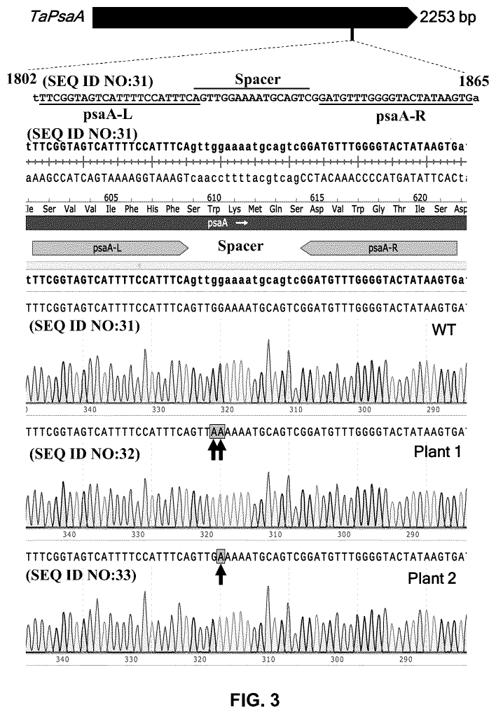

FIG. 4 is a schematic illustrating the structure of mitochondrial ATP6 (“mitoATP6”) targeted by a cytosine base editor and a representative of Sanger sequencing chromatogram indicating C·G to T·A conversion in mitoATP6 in rice transgenic plants. Rice transgenic plants were generated by introducing DNA constructs expressing TALCDAs (OsATP6-L1 and OsATP6-R1) targeting the mitoATP6 gene and subjected to DNA extraction. PCR-amplicons from the targeted region were subjected to Sanger sequencing. Arrows indicate the nucleotide conversions.

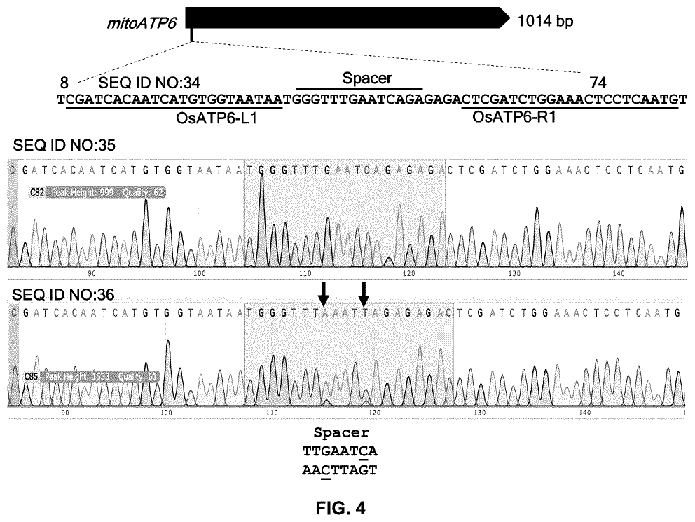

FIG. 5 is a schematic illustrating the structure of mitochondrial ATP6 (“mitoATP6”) targeted by a cytosine base editor and a representative of Sanger sequencing chromatogram indicating C·G to T·A conversion in mitoATP6 in rice transgenic plants. Rice transgenic plants were generated by introducing DNA constructs expressing TALCDAs (OsATP6-L2 and OsATP6-R2) targeting the mitoATP6 gene and subjected to DNA extraction. PCR-amplicons from the targeted region were subjected to Sanger sequencing. Arrows indicate the nucleotide conversions.

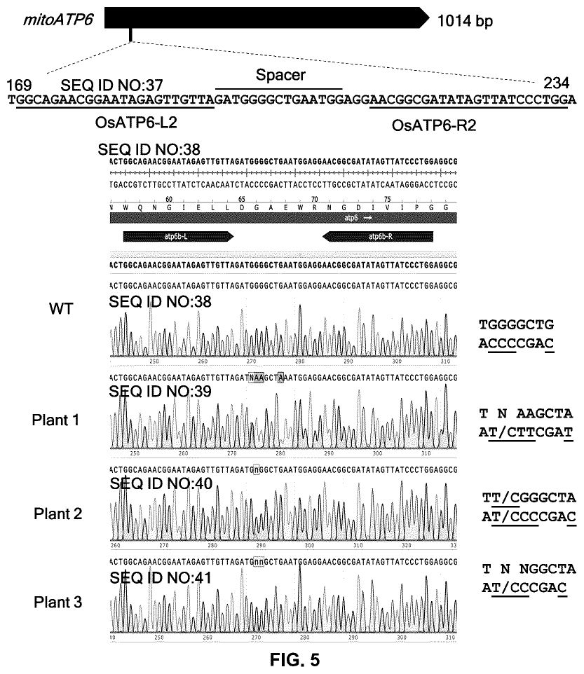

FIG. 6 is a schematic illustrating the structure of PsbA targeted by a cytosine base editor and a representative of Sanger sequencing chromatogram indicating C·G to T·A conversion in the PsbA gene in transgenic maize plants. Maize plants were generated by introducing the DNA constructs expressing TALCDAs targeting the PsbA gene. Genomic DNA samples were extracted from leaves of individual transgenic plants. PCR-amplicons from the targeted region were subjected to Sanger sequencing. Arrows indicate the nucleotide conversions. The spacer region is shaded.

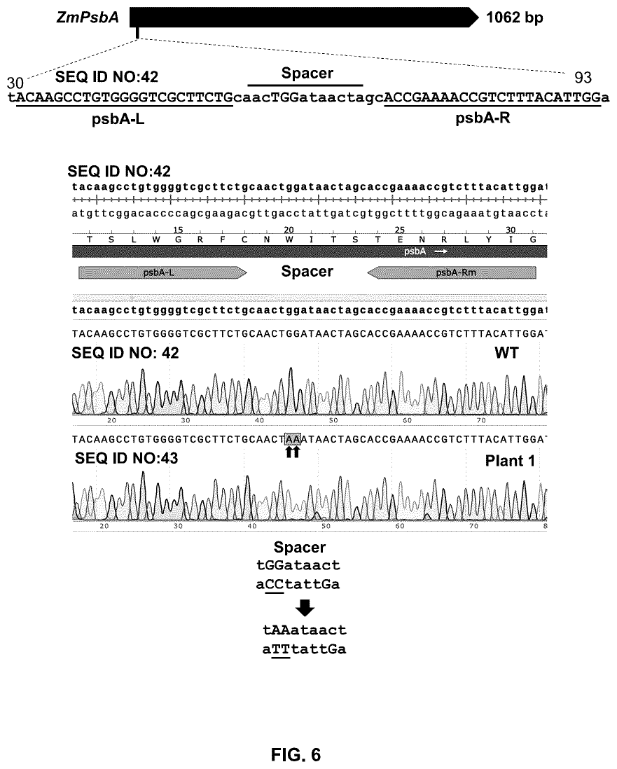

FIG. 7 is a schematic illustrating a nucleic acid encoding a cytosine base editor and a selection marker (e.g., Hyg, Bar, or GFP) which when expressed provides a recombinant fusion protein encoding a deaminase (e.g., “TAL-SCP”). Upon targeting to a plant chloroplast (“cp”) or plant mitochondria (“mt”), the TAL-SPC binds a target DNA to mediate gene editing at the target DNA site.

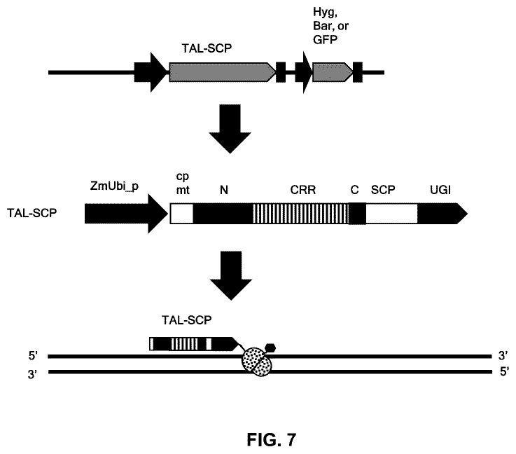

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid synthesis. The techniques and procedures are generally performed according to conventional methods in the art and various general references. Although any methods and materials similar to or equivalent to those described herein can be used in the practice or testing of the present disclosure, the preferred methods and materials are described herein. Moreover, reference to an element by the indefinite article “a” or “an” does not exclude the possibility that more than one element is present, unless the context clearly requires that there be one and only one element. The indefinite article “a” or “an” thus usually includes “at least one.”

As used herein, a “nucleic acid” sequence means a DNA or RNA sequence, and a mix of DNA and RNA. “Nucleic acid” also encompasses sequences that include natural nucleotides and known base analogues of DNA and RNA such as 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxy methylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine. Nucleic acids may be of genomic or synthetic origin and may be single-stranded, double-stranded, and triple-stranded. Polynucleotides and oligonucleotides are within the scope of nucleic acids.

Nucleic acids used in the methods of the present disclosure preferably are codon optimized for use in plants and, in particular, plant chloroplasts and plant mitochondria. As known in the art, “codon optimized” is a process used to improve gene expression and increase the translational efficiency of a gene of interest by accommodating codon bias of the host organism. Codon optimization can be performed using commercially available tools (e.g., Codon Optimization Tool from Integrated DNA Technologies).

For nucleotide (and nucleic acid) sequences, “variant” refers to a similar but not identical nucleotide sequence to a reference nucleotide sequence. For nucleotide sequences, a variant includes a nucleotide sequence having deletions (i.e., truncations) at the 5′ and/or 3′ end, deletions and/or additions of one or more nucleotides at one or more internal sites compared to the nucleotide sequence of the reference nucleic acid molecules as described herein; and/or substitution of one or more nucleotides at one or more sites compared to the nucleotide sequence of the reference nucleic acid molecules described herein. In some embodiments, variants are constructed in a manner to maintain the open reading frame.

Naturally occurring allelic variants can be identified by using well-known molecular biology techniques such as, for example, polymerase chain reaction (PCR) and hybridization techniques. Variant nucleotide sequences also can include synthetically derived sequences, such as those generated, for example, by site-directed mutagenesis but which still provide a functionally active modified protein. Generally, variants of a nucleotide sequence of the reference nucleic acid molecules as described herein will have at least about 70%, 75%, 80%, 85%, 90%, 91%, 92%, 93%, 94%, 95%, 96%, 97%, 98%, 99% or more sequence identity to the nucleotide sequence of the reference nucleic acid molecules as determined by sequence alignment programs and parameters as described elsewhere herein.

Variants of the reference nucleic acid molecules described herein also can be evaluated by comparing the percent sequence identity between the polypeptide encoded by a variant and the polypeptide encoded by the reference nucleic acid molecule. Thus, for example, an isolated nucleic acid molecule can be one that encodes a polypeptide with a given percent sequence identity to the polypeptide of interest. Percent sequence identity between any two polypeptides can be calculated using sequence alignment programs and parameters described elsewhere herein. Where any given pair of polynucleotides of the present disclosure is evaluated by comparison of the percent sequence identity shared by the two polypeptides they encode, the percent sequence identity between the two encoded polypeptides can be at least about 70%, 75%, 80%, 85%, 90%, 91%, 92%, 93%, 94%, 95%, 96%, 97%, 98%, 99% or more sequence identity.

Determining percent sequence identity between any two sequences can be accomplished using a mathematical algorithm as described in Myers & Miller (1988) CABIOS 4:11-17; the local alignment algorithm of Smith et al. (1981) Adv. Appl. Math. 2:482-489; the global alignment algorithm of Needleman & Wunsch (1970) J. Mol. Biol. 48:443-453; the search-for-local alignment method of Pearson & Lipman (1988) Proc. Natl. Acad. Sci. USA 85:2444-2448; and the algorithm of Karlin & Altschul (1990) Proc. Natl. Acad. Sci. USA 87:2264-2268, modified as in Karlin & Altschul (1993) Proc. Natl. Acad. Sci. USA 90:5873-5877.

As used herein, “recombinant,” when used in connection with a nucleic acid molecule, refers to a molecule that has been created or modified through deliberate human intervention by genetic engineering. For example, a recombinant nucleic acid molecule is one having a nucleotide sequence that has been modified to include an artificial nucleotide sequence or to include some other nucleotide sequence that is not present within its native (non-recombinant) form. Further, a recombinant nucleic acid molecule has a structure that is not identical to that of any naturally occurring nucleic acid molecule or to that of any fragment of a naturally occurring genomic nucleic acid molecule spanning more than one gene. A recombinant nucleic acid molecule also includes a nucleic acid molecule having a sequence of a naturally occurring genomic or extrachromosomal nucleic acid molecule, but which is not flanked by the coding sequences that flank the sequence in its natural position; a nucleic acid molecule incorporated into a construct, expression cassettes or vectors, or into a host cell's genome such that the resulting polynucleotide is not identical to any naturally occurring vector or genomic DNA; a separate nucleic acid molecule such as a cDNA, a genomic fragment, a fragment produced by polymerase chain reaction (PCR) and other amplification methods or a restriction fragment; and a recombinant nucleic acid molecule having a nucleotide sequence that is part of a hybrid gene (i.e., a gene encoding a fusion protein). As such, a recombinant nucleic acid molecule can be modified (chemically or enzymatically) or unmodified DNA or RNA, whether fully or partially single-stranded or double-stranded or even triple-stranded.

Methods for synthesizing nucleic acid molecules are well known in the art, such as cloning and digestion of the appropriate sequences in genetic engineering, as well as direct chemical synthesis. Methods of cloning nucleic acid molecules are described, for example, in Ausubel et al. (1995), supra; Copeland et al. (2001) Nat. Rev. Genet. 2:769-779; PCR Cloning Protocols, 2nd ed. (Chen & Janes eds., Humana Press 2002); and Sambrook & Russell (2001), supra. Methods of direct chemical synthesis of nucleic acid molecules can be done using the phosphotriester methods of Reese (1978) Tetrahedron 34:3143-3179 and Narang et al. (1979) Methods Enzymol. 68:90-98; the phosphodiester method of Brown et al. (1979) Methods Enzymol. 68:109-151; the diethylphosphoramidate method of Beaucage et al. (1981) Tetrahedron Lett. 22:1859-1862; and the solid support methods of Fodor et al. (1991) Science 251:767-773; Pease et al. (1994) Proc. Natl. Acad. Sci. USA 91:5022-5026; and Singh-Gasson et al. (1999) Nature Biotechnol. 17:974-978; as well as U.S. Pat. No. 4,485,066. See also, Peattie (1979) Proc. Natl. Acad. Sci. USA 76:1760-1764; as well as EP Patent No. 1721908; Int'l Patent Application Publication Nos. WO 2004/022770 and WO 2005/082923; US Patent Application Publication No. 2009/0062521; and U.S. Pat. Nos. 6,521,427; 6,818,395 and 7,521,178.

Methods of mutating and altering nucleotide sequences, as well as DNA shuffling, are well known in the art. See, Crameri et al. (1997) Nature Biotech. 15:436-438; Crameri et al. (1998) Nature 391:288-291; Kunkel (1985) Proc. Natl. Acad. Sci. USA 82:488-492; Kunkel et al. (1987) Methods in Enzymol. 154:367-382; Moore et al. (1997) J. Mol. Biol. 272:336-347; Stemmer (1994) Proc. Natl. Acad. Sci. USA 91:10747-10751; Stemmer (1994) Nature 370:389-391; Zhang et al. (1997) Proc. Natl. Acad. Sci. USA 94:4504-4509; and Techniques in Molecular Biology (Walker & Gaastra eds., MacMillan Publishing Co. 1983) and the references cited therein; as well as U.S. Pat. Nos. 4,873,192; 5,605,793 and 5,837,458. As such, the nucleic acid molecules as described herein can have many modifications.

Methods of introducing DNA molecules into plant cells are well known to those of skill in the art. Suitable methods include bacterial infection, binary BAC vectors, and direct delivery of DNA (e.g., by PEG-mediated transformation, desiccation/inhibition-mediated DNA uptake, electroporation, agitation with silicon carbide fibers, and acceleration of DNA coated particles).

Vectors useful for expression of nucleic acids in higher plants are well known in the art and include vectors derived from the Ti plasmid of Agrobacterium tumefaciens and the pCaMVCN transfer control vector.

As used herein, “coupled” (used interchangeably herein with “operably linked”) refers to being joined as part of the same molecule. The term “operably linked” refers to a first DNA molecule joined to a second DNA molecule, wherein the first and second DNA molecules are so arranged that the first DNA molecule affects the function of the second DNA molecule. The two DNA molecules may or may not be part of a single contiguous DNA molecule and may or may not be adjacent. “Operably linked” refers to two or more nucleic acid sequence elements that are physically linked and are in a functional relationship with each other. For instance, a promoter is operably linked to a coding sequence if the promoter is able to initiate or regulate the transcription or expression of a coding sequence, in which case the coding sequence should be understood as being “under the control of” the promoter. Generally, when two nucleic acid sequences are operably linked, they will be in the same orientation and usually also in the same reading frame. They usually will be essentially contiguous, although this may not be required.

“Fusion protein”, as used herein, a protein consisting of at least two domains that are encoded by separate genes (or portions of genes) that have been joined so that they are transcribed and translated as a single unit, producing a single polypeptide.

Disclosed herein are cytosine base editors tailored for chloroplast and mitochondrial genomes in plants by using plant-specific chloroplast and mitochondrial targeting peptides, a codon-optimized TALE, split-halves of DddA and, optionally, at least one UGI. The systems of the present disclosure include a serial of DNA vectors and protocols to use them.

### Recombinant Fusion Proteins and Nucleic Acids Encoding Recombinant Fusion Proteins

In one aspect, the present disclosure is directed to a recombinant fusion protein including a targeting peptide selected from the group consisting of a plant chloroplast targeting peptide and a plant mitochondrial targeting peptide, a TALE array protein, and a deaminase. The recombinant fusion protein can further include at least one uracil glycosylase inhibitor (UGI).

A particularly suitable recombinant fusion protein includes a targeting peptide selected from the group consisting of a plant chloroplast targeting peptide and a plant mitochondrial targeting peptide, a TALE array protein, a deaminase, and an uracil glycosylase inhibitor.

In one aspect, the present disclosure is directed to a recombinant nucleic acid molecule encoding a recombinant fusion protein comprising a targeting peptide selected from the group consisting of a plant chloroplast targeting peptide and a plant mitochondrial targeting peptide, a TALE array protein, and a deaminase. In one embodiment, the nucleic acid molecule includes a nucleic acid sequence encoding a targeting peptide selected from the group consisting of a plant chloroplast targeting peptide and a plant mitochondrial targeting peptide operably linked to a nucleic acid sequence encoding a transcription activator-like effector (TALE) array protein operably linked to a nucleic acid sequence encoding a deaminase.

In one aspect, the present disclosure is directed to a recombinant nucleic acid molecule encoding a recombinant fusion protein comprising a targeting peptide selected from the group consisting of a plant chloroplast targeting peptide and a plant mitochondrial targeting peptide, a TALE array protein, a deaminase, and an uracil glycosylase inhibitor. In one embodiment, the nucleic acid molecule includes a nucleic acid sequence encoding a chloroplast targeting peptide operably linked to a nucleic acid sequence encoding a transcription activator-like effector (TALE) array protein operably linked to a nucleic acid sequence encoding a deaminase operably linked to a nucleic acid sequence encoding an uracil glycosylase inhibitor.

In one embodiment, the nucleic acid molecule includes a nucleic acid sequence encoding a mitochondria targeting peptide operably linked to a nucleic acid sequence encoding a transcription activator-like effector (TALE) array protein operably linked to a nucleic acid sequence encoding a deaminase operably linked to a nucleic acid sequence encoding an uracil glycosylase inhibitor. Particularly suitable nucleic acid molecules encoding a recombinant fusion protein comprising a targeting peptide selected from the group consisting of a plant chloroplast targeting peptide and a plant mitochondrial targeting peptide, a TALE array protein, a deaminase, and an uracil glycosylase inhibitor include SEQ ID NO:1, SEQ ID NO:2, SEQ ID NO:3, SEQ ID NO:4, SEQ ID NO:5, and SEQ ID NO:6.

### Chloroplast and Mitochondrial Targeting Peptides

Constructs (backbone vectors that will be used to construct specific TALE deaminases for plant chloroplast and mitochondrial genomes) are distinguished by the targeting signals at their N-termini. The constructs include nucleic acids encoding a chloroplast targeting peptide or a mitochondria targeting peptide to direct the fusion protein containing the TALE deaminases into the target organelles (i.e., chloroplast targeting signal directs the TALE deaminase to chloroplasts and mitochondrial targeting signal directs the TALE deaminase to mitochondria).

Examples of chloroplast targeting peptides include those associated with the small subunit (SSU) of ribulose-1,5,-bisphosphate carboxylase, ferredoxin, ferredoxin oxidoreductase, the light-harvesting complex protein I and protein II, thioredoxin F, and enolpyruvyl shikimate phosphate synthase (EPSPS). Non-chloroplast proteins (e.g., deaminase, transcription activator-like effector (TALE) array protein, and uracil glycosylase inhibitor) are targeted to the chloroplast by the expression of a heterologous chloroplast targeting peptide fused to the non-chloroplast proteins. A particularly suitable nucleotide sequence encoding a chloroplast targeting peptide is provided in SEQ ID NO:7.

Suitable examples of mitochondria targeting peptides are described in Sjoling and Glaser (“Mitochondrial targeting peptides in plants,” Trends in Plant Science Apr. 1, 1998, 3(4):136-140), Huang et al. (Plant Physiology, July 2009, 150:1272-1285), Murcha et al. (J. Experimental Botony, Oct. 16, 2014, 65(22): 6301-6335), which are incorporated herein by reference in its entirety, Mitochondrial-Targeting Signal 1 (“MITS1”) described in Chatre et al. (J Exp Bot. 2009 March; 60(3): 741-749). Non-mitochondria proteins (e.g., deaminase, transcription activator-like effector (TALE) array protein, and uracil glycosylase inhibitor) are targeted to the plant mitochondria by the expression of a heterologous mitochondria targeting peptide fused to the non-mitochondrial proteins. A particularly suitable nucleotide sequence encoding a mitochondria targeting peptide is provided in SEQ ID NO:8. A particularly suitable mitochondrial targeting peptide is obtained from Nicotiana plubaginifolia ATP2-1 gene for mitochondrial ATP synthase beta subunit (SEQ ID NO:27).

### TALE

TALEs (also interchangeably referred to herein as “TALE array protein”) of bacterial origin recognize DNA sequences of target sites following a TALE DNA recognition code, i.e., one modular repeat of 34 amino acids corresponds to one nucleotide and four predominant repeats recognize four nucleotides respectively (Boch et al. 2009; Moscou et al. 2009). Specifically, two amino acids at the position 12 and 13, so-called repeat variable di-residues (RVDs), of the 34 amino acids determine the specificity of nucleotide recognition, e.g., NI, HD, NG and NN corresponding to A, C, T, G for DNA binding. Therefore, the DNA binding domains of designer TALEs can be modularly assembled based on the TALE DNA recognition code and the preselected genomic sequences (Li et al. 2012).

### DNA Deaminase

Suitable deaminases include SCP1.201-like DNA deaminases. Particularly suitable DNA deaminases are double-stranded DNA deaminases. Particularly suitable double-stranded DNA deaminases are double-stranded DNA cytidine deaminases. Particularly suitable deaminases include DddA, SCPa, SCPb, and SCPc. A particularly suitable DddA is NCBI accession code WP_080324253.1. A particularly suitable SCPa is NCBI accession code WP_091452319.1 obtained from Actinokineospora iranica. A particularly suitable SCPb is NCBI accession code WP_228772027.1 obtained from Actinokineospora iranica. A particularly suitable SCPc is NCBI accession code WP_021798742.1 obtained from Propionibacterium acidifaciens. In some embodiments, the double stranded DNA deaminase is a full-length deaminase. The full-length deaminase binds a target DNA to execute deamination activity. In some embodiments, the double stranded DNA deaminase is a split-deaminase. As used herein, a “split-deaminase” is a deaminase that includes less than the full-length protein. For example, a split-deaminase can have an N-terminal truncation. Another example of a split-deaminase has a C-terminal truncation. Particularly suitable split-deaminases include a G1333 split-DddA and a G1397 split-DddA (both named to reflect the last amino acid residue of a N-terminal truncated DddA). When combined, the N-terminal split-deaminase and the C-terminal split-deaminase reconstitute deamination activity when adjacently assembled on a target DNA. A particularly suitable DddA consists of N-terminal 108 amino acids and C-terminal 30 amino acids of the DddA domain (amino acid position 1290 to 1427 of the RHS domain-containing protein from Burkholderia cenocepacia with the NCBI accession number WP_080324253). As used herein CDA-L refers to the N-terminal split-cytosine deaminase domain and CDA-R refers to the C-terminal split-cytosine deaminase domain.

### Uracil Glycosylase Inhibitor

The recombinant fusion protein can further include at least one uracil glycosylase inhibitor (UGI, also known as uracil-DNA glycosylase inhibitor). UGI binds uracil glycosylase to inhibit the removal of uracil residues from DNA by the uracil-excision repair system. Uracil-DNA glycosylase functions to prevent mutagenesis by eliminating uracil from DNA by cleaving the N-glycosidic bond and initiating base-excision repair. In one embodiment, the UGI is located at the N-terminus of the recombinant fusion protein. In one embodiment, two UGI are located at the N-terminus of the recombinant fusion protein. In another embodiment, the UGI is located at the C-terminus of the recombinant fusion protein. In one embodiment, two UGI are located at the C-terminus of the recombinant fusion protein. A suitable uracil glycosylase inhibitor has the amino acid sequence of UniProtKB-P14739 (UNGI_BPPB2) (SEQ ID NO:9).

### Spacers

The recombinant fusion protein can further include at least one spacer. Spacers can range from 2 amino acid residues to 40 amino acid residues including 2 amino acid spacers, 4 amino acid spaces, 10, amino acid spacers, 16 amino acid spacers, and 32 amino acid. Spacers are preferably positioned between each of the protein domains forming the fusion protein. For example, a spacer is included between the chloroplast targeting peptide and the TALE, between the TALE and the deaminase, and between the deaminase and the UGI. Suitable amino acid spacers include glycine and glycine-serine spacers. Suitable amino acid spacers include (GG)ₙ, (GS)ₙ, (SG)ₙ, (SGGS)ₙ, wherein n is an integer ranging from 1 to 100. For example, (GG)₁ is a 2 glycine amino acid spacer. Similarly, (GG)₂ is a GGGG amino acid spacer.

### Selection Markers

In some embodiments, the recombinant fusion protein of the present disclosure can further include a selection marker. Suitable selection markers are known in the art such as antibiotic selection markers, herbicide selection markers, visual selection markers, and combinations thereof Antibiotic selection markers include hygromycin phosphotransferase, neomycin (neomycin phosphotransferase II and III), bleomycin, and aminoglycoside adenyltransferase, for example. Herbicide selection markers include bar (phosphinothricin acetyl transferase), enolpyruvyl shikimate phosphate synthase, acetolactase synthase, glyphosate oxidoreductase, and bromoxynil nitrilase, for example. Visual selection markers include green fluorescence, red fluorescence, yellow fluorescence, and cyan fluorescence such as GFP, eGFP, G3GFP, sfGFP, DsRed2, mRuby2, mCherry, tdTomato, Clover, EYFP, YPet, mVenus, mCerulean, and ECFP, for example.

### Nucleic Acid Constructs

Nucleic acid constructs of the present disclosure include a vector, in particular a plasmid, cosmid, phage, linear nucleotide sequences, circular nucleotide sequence, of a single or double stranded DNA or RNA, derived from any source, in which a number of nucleotide sequences have been joined or recombined into a unique construction that is capable of introducing any one of the nucleotide sequences described herein in sense or antisense orientation into a cell, in particular a plant cell. The choice of vector depends on the recombinant procedures followed and the host cell used. The vector may be an autonomously replicating vector or may replicate together with the chromosome into which it has been integrated. The vector can further include a selection marker as described herein. Useful markers are dependent on the host cell of choice and are well known to persons skilled in the art. In case the protein is to be obtained from leaves or roots, infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid. Agrobacterium-based plasmid vectors are suitable for stable transformation of nucleic acid constructs in a plant genome. The choice of the transformation vector is dependent on the transformation procedure and the host cell. Binary Ti vectors which can be used for Agrobacterium-mediated gene transfer include pBIN19, pC22, pGA482 and pPCV001.

The nucleic acid constructs include a first nucleic acid encoding a recombinant protein including a targeting peptide operably linked to a nucleic acid sequence encoding a TALE array protein operably linked to a nucleic acid sequence encoding a deaminase. The nucleic acid constructs can include a first nucleic acid encoding a recombinant protein including a targeting peptide operably linked to a nucleic acid sequence encoding a TALE array protein operably linked to a nucleic acid sequence encoding a deaminase operably linked to a nucleic acid sequence encoding an uracil glycosylase inhibitor. The nucleic acid constructs can include a first nucleic acid encoding a recombinant protein including a targeting peptide operably linked to a nucleic acid sequence encoding a TALE array protein operably linked to a nucleic acid sequence encoding a first split-half deaminase and a second nucleic acid encoding a recombinant protein including a targeting peptide operably linked to a nucleic acid sequence encoding a TALE array protein operably linked to a nucleic acid sequence encoding a second split-half deaminase. The nucleic acid constructs can include a first nucleic acid encoding a recombinant protein including a targeting peptide operably linked to a nucleic acid sequence encoding a TALE array protein operably linked to a nucleic acid sequence encoding a first split-half deaminase operably linked to a nucleic acid sequence encoding an uracil glycosylase inhibitor and a second nucleic acid encoding a recombinant protein including a targeting peptide operably linked to a nucleic acid sequence encoding a TALE array protein operably linked to a nucleic acid sequence encoding a second split-half deaminase operably linked to a nucleic acid sequence encoding an uracil glycosylase inhibitor. The nucleic acid constructs can optionally further include a nucleic acid encoding a selectable marker.

In one aspect, the present disclosure is directed to a nucleic acid provided in SEQ ID NO:1.

In one aspect, the present disclosure is directed to a nucleic acid provided in SEQ ID NO:2.

In one aspect, the present disclosure is directed to a nucleic acid provided in SEQ ID NO:3.

In one aspect, the present disclosure is directed to a nucleic acid provided in SEQ ID NO:4.

In one aspect, the present disclosure is directed to a nucleic acid provided in SEQ ID NO:5.

In one aspect, the present disclosure is directed to a nucleic acid provided in SEQ ID NO:6.

### Promoters

Nucleic acids can further include plant and tissue specific promoters. Suitable promoters include constitutively active promoters and inducible promoters. Promotors as used herein include plant-specific, tissue-specific, tissue-preferred, cell-type-specific, inducible and constitutive promotors. Tissue-specific promotors are promoters that initiate transcription only in certain tissues and refer to a sequence of DNA that provides recognition signals for RNA polymerase and/or other factors required for transcription to begin, and/or for controlling expression of the coding sequence precisely within certain tissues or within certain cells of that tissue. Expression in a tissue specific manner may be only in individual tissues or in combinations of tissues. Tissue-specific promoters are reviewed by Edwards, J. W. & Cornzzi, G. M., Annu Rev. Genet. 24, 275-303 (1990) and include embryo-specific promotors such as the promoters of the embryonic storage proteins soybean 0-conglycinin gene, legumin genes from common bean, β phaseolin gene and napin and cruciferin genes from rapeseed, endosperm-specific promotors such as the promoters of maize zein genes, wheat glutenin genes and barley hordein genes, fruit-specific promotors such as the promotor of the tomato ethylene-responsive E8 gene, tuber-specific promotors such as the class-I patatin promotor of potato and leaf-specific promotors such as the promotors of ribulose-1,5-biphosphate carboxylase small subunit gene and the chlorophyll a/b binding protein gene.

Suitable promoters include inducible promoters that are capable of activating transcription of one or more DNA sequences or genes in response to an inducer. Inducers known in the art include high salt concentrations, cold, heat or toxic elements and include pathogens or disease agents such as viruses. Inducers include chemical agents such as herbicides, proteins, growth regulators, metabolites or phenolic compounds. The inducer can also be an illumination agent such as darkness and light at various modalities including wavelength, intensity, fluence, direction and duration. Activation of an inducible promoter is established by application of the inducer. Generally, inducible promotors include the hsp70 heat shock promoter of Drosphilia melanogaster, a cold inducible promoter from Brassica napus and an alcohol dehydrogenase promoter that is induced by ethanol. Specific plant inducible promotors include the tetracycline-inducible promotor and the α-amylase promotor.

Suitable promoters also include constitutive promoters that are active under many environmental conditions and in many different tissue types. Constitutive promotors include the 35S promotor or 19S promotor of the cauliflower mosaic virus (CaMV), the ubiquitin promotor, the coat promoter of TMV, the cassava vein mosaic virus promotors (CsVMV), the rice actin-I promotor and regulatory regions associated with Agrobacterium genes, such as nopaline synthase (Nos), mannopine synthase (Mas) or octopine synthase (Ocs).

### Method of Gene Editing in Plant Chloroplasts

In one aspect, the present disclosure is directed to a method of editing a plant chloroplast nucleic acid. In one embodiment, the method includes providing a recombinant fusion protein comprising a plant chloroplast targeting peptide, a TALE array protein, and a deaminase, wherein the recombinant fusion protein localizes to a chloroplast and forms a complex with a target chloroplast double-stranded nucleic acid and catalyzes a C·G to T·A conversion in the target chloroplast double-stranded nucleic acid. In another embodiment, the recombinant fusion protein can further comprise a selectable marker. In another embodiment, the method includes providing a recombinant fusion protein comprising a plant chloroplast targeting peptide, a TALE array protein, a deaminase and at least one UGI, wherein the recombinant fusion protein localizes to a chloroplast and forms a complex with a target chloroplast double-stranded nucleic acid and catalyzes a C·G to T·A conversion in the target chloroplast double-stranded nucleic acid. In another embodiment, the recombinant fusion protein can further comprise a selectable marker.

The recombinant protein can be provided by introducing a nucleic acid encoding the recombinant protein. In one embodiment, the nucleic acid includes a nucleic acid encoding a recombinant protein including a chloroplast targeting peptide operably linked to a nucleic acid sequence encoding a TALE array protein operably linked to a nucleic acid sequence encoding a deaminase. The nucleic acid can further include a nucleic acid sequence encoding at least one uracil glycosylase inhibitor. The nucleic acid can further include a nucleic acid sequence encoding a selectable marker. In one embodiment, the nucleic acid includes a first nucleic acid encoding a recombinant protein including a chloroplast targeting peptide operably linked to a nucleic acid sequence encoding a TALE array protein operably linked to a nucleic acid sequence encoding a first split-half deaminase and a second nucleic acid encoding a recombinant protein including a chloroplast targeting peptide operably linked to a nucleic acid sequence encoding a TALE array protein operably linked to a nucleic acid sequence encoding a second split-half deaminase. In one embodiment, the nucleic acid includes a first nucleic acid encoding a recombinant protein including a chloroplast targeting peptide operably linked to a nucleic acid sequence encoding a TALE array protein operably linked to a nucleic acid sequence encoding a first split-half deaminase operably linked to a nucleic acid sequence encoding at least one uracil glycosylase inhibitor and a second nucleic acid encoding a recombinant protein including a chloroplast targeting peptide operably linked to a nucleic acid sequence encoding a TALE array protein operably linked to a nucleic acid sequence encoding a second split-half deaminase operably linked to a nucleic acid sequence encoding at least one uracil glycosylase inhibitor. The nucleic acid can further include a nucleic acid sequence encoding a selectable marker.

In one embodiment, the method includes providing a first recombinant fusion protein comprising a plant chloroplast targeting peptide, a TALE array protein, and a split-half deaminase, wherein the recombinant fusion protein localizes to a chloroplast; providing a second recombinant fusion protein comprising a plant chloroplast targeting peptide, a TALE array protein, and a split-half deaminase; wherein the first recombinant fusion protein and the second recombinant fusion protein localizes to a chloroplast and forms a complex with a target chloroplast double-stranded nucleic acid and catalyzes a C·G to T·A conversion in the target chloroplast double-stranded nucleic acid. The nucleic acid can further include a nucleic acid sequence encoding a selectable marker. In another embodiment, the method includes providing a first recombinant fusion protein comprising a plant chloroplast targeting peptide, a TALE array protein, a split-half deaminase, and at least one UGI, wherein the recombinant fusion protein localizes to a chloroplast; providing a second recombinant fusion protein comprising a plant chloroplast targeting peptide, a TALE array protein, and a split-half deaminase; wherein the first recombinant fusion protein and the second recombinant fusion protein localizes to a chloroplast and forms a complex with a target chloroplast double-stranded nucleic acid and catalyzes a C·G to T·A conversion in the target chloroplast double-stranded nucleic acid. The nucleic acid can further include a nucleic acid sequence encoding a selectable marker.

### Method of Gene Editing in Plant Mitochondria

In one aspect, the present disclosure is directed to a method of editing a plant mitochondria nucleic acid. In one embodiment, the method includes providing a recombinant fusion protein including a plant mitochondria targeting peptide, a TALE array protein, and a deaminase, wherein the recombinant fusion protein localizes to a mitochondria and forms a complex with a target mitochondria double-stranded nucleic acid and catalyzes a C·G to T·A conversion in the target mitochondria double-stranded nucleic acid. The nucleic acid can further include a nucleic acid sequence encoding at least one UGI. The nucleic acid can further include a nucleic acid sequence encoding a selectable marker. In another embodiment, the method includes providing a recombinant fusion protein comprising a plant mitochondria targeting peptide, a TALE array protein, a deaminase and at least one UGI, wherein the recombinant fusion protein localizes to a mitochondria and forms a complex with a target mitochondria double-stranded nucleic acid and catalyzes a C·G to T·A conversion in the target mitochondria double-stranded nucleic acid. The nucleic acid can further include a nucleic acid sequence encoding a selectable marker.

In one embodiment, the method includes providing a first recombinant fusion protein including a plant mitochondria targeting peptide, a TALE array protein, and a split-half deaminase, wherein the recombinant fusion protein localizes to a mitochondria; providing a second recombinant fusion protein comprising a plant mitochondria targeting peptide, a TALE array protein, and a split-half deaminase; wherein the first recombinant fusion protein and the second recombinant fusion protein localizes to a mitochondria and forms a complex with a target mitochondria double-stranded nucleic acid and catalyzes a C·G to T·A conversion in the target mitochondria double-stranded nucleic acid. The nucleic acid can further include a nucleic acid sequence encoding at least one UGI. The nucleic acid can further include a nucleic acid sequence encoding a selectable marker. In another embodiment, the method includes providing a first recombinant fusion protein comprising a plant mitochondria targeting peptide, a TALE array protein, a split-half deaminase, and at least one UGI, wherein the recombinant fusion protein localizes to a mitochondria; providing a second recombinant fusion protein comprising a plant mitochondria targeting peptide, a TALE array protein, and a split-half deaminase; wherein the first recombinant fusion protein and the second recombinant fusion protein localizes to a mitochondria and forms a complex with a target mitochondria double-stranded nucleic acid and catalyzes a C·G to T·A conversion in the target mitochondria double-stranded nucleic acid. The nucleic acid can further include a nucleic acid sequence encoding a selectable marker.

The recombinant protein can be provided by introducing a nucleic acid encoding the recombinant protein. In one embodiment, the nucleic acid includes a nucleic acid encoding a recombinant protein including a mitochondria targeting peptide operably linked to a nucleic acid sequence encoding a TALE array protein operably linked to a nucleic acid sequence encoding a deaminase. The nucleic acid can further include a nucleic acid sequence encoding at least one uracil glycosylase inhibitor. The nucleic acid can further include a nucleic acid sequence encoding a selectable marker. In one embodiment, the nucleic acid includes a first nucleic acid encoding a recombinant protein including nucleic acid encoding a mitochondria targeting peptide operably linked to a nucleic acid sequence encoding a TALE array protein operably linked to a nucleic acid sequence encoding a first split-half deaminase and a second nucleic acid encoding a recombinant protein including a mitochondria targeting peptide operably linked to a nucleic acid sequence encoding a TALE array protein operably linked to a nucleic acid sequence encoding a second split-half deaminase. The first nucleic acid and/or the second nucleic acid can further include a nucleic acid sequence encoding at least one UGI. The first nucleic acid and/or the second nucleic acid can further include a nucleic acid sequence encoding a selectable marker. In one embodiment, the nucleic acid includes a first nucleic acid encoding a recombinant protein including a mitochondria targeting peptide operably linked to a nucleic acid sequence encoding a TALE array protein operably linked to a nucleic acid sequence encoding a first split-half deaminase operably linked to a nucleic acid sequence encoding at least one uracil glycosylase inhibitor and a second nucleic acid encoding a recombinant protein including a mitochondria targeting peptide operably linked to a nucleic acid sequence encoding a TALE array protein operably linked to a nucleic acid sequence encoding a second split-half deaminase operably linked to a nucleic acid sequence encoding at least one uracil glycosylase inhibitor. The first nucleic acid and/or the second nucleic acid can further include a nucleic acid sequence encoding a selectable marker.

The nucleic acids and recombinant proteins of the present disclosure can be introduced to a plant cell by introducing a first nucleic acid that encodes a recombinant protein having a chloroplast or mitochondria targeting peptide, a TALE array protein, a first split-half deaminase, and at least one uracil glycosylase inhibitor and a second nucleic acid that encodes a recombinant protein having a chloroplast or mitochondria targeting peptide, a TALE array protein, a first split-half deaminase, and at least one uracil glycosylase inhibitor, wherein the first nucleic acid and the second nucleic acid are provided separately. The first nucleic acid and/or the second nucleic acid can further include a nucleic acid sequence encoding a selectable marker. In a preferred embodiment, a first nucleic acid that encodes a recombinant protein having a chloroplast or mitochondria targeting peptide, a TALE array protein, a first split-half deaminase, and at least one uracil glycosylase inhibitor and a second nucleic acid that encodes a recombinant protein having a chloroplast or mitochondria targeting peptide, a TALE array protein, a first split-half deaminase, and at least one uracil glycosylase inhibitor are provided in the same nucleic acid (e.g., vector). In this embodiment, transcription of the single vector results in the production of both fusion proteins. Because the first split-half and the second split-half deaminases must reconstitute at the target DNA site to be active, expression from the same nucleic acid construct produces equimolar amounts of each fusion protein.

Editing of specific sites in either chloroplasts or mitochondria is defined by the TALE deaminase. One or a pair of site-specific TALE deaminases are engineered based on the DNA sequences of that specific target site. Each construct carries out a specific gene edit based on design of the construct. For example, a construct may be designed to create a stop codon in mitochondria of maize. That same construct would not be able to create the stop codon in the mitochondria of rice or a stop codon in the chloroplast of maize or make a different variation in the DNA that could not be created by a cytidine deamination at that site (C to T).

Conserved function of chloroplast targeting peptides and mitochondrial targeting peptides among plant species permits the constructs to function in a diverse range of plant species. The methods can be performed in any plant including monocots and dicots. The method is particularly suitable for use in crop plants. Particularly desirable plants are rice, corn, soybeans, sorghum, and wheat.

The methods of gene editing in plant chloroplasts and plant mitochondria are suitable for cells, tissues, organs, and progeny of the plants. Plant tissues and cells of particular interest include protoplasts, calli, roots, tubers, seeds, stems, leaves, seedlings, embryos, and pollen.

Disclosed herein are cytosine base editors tailored for chloroplast and mitochondrial genomes in plants by using plant-specific targeting peptides and codon-optimized TALE, a deaminase and, optionally, at least one UGI. Also disclosed herein are cytosine base editors tailored for chloroplast and mitochondrial genomes in plants by using plant-specific targeting peptides and codon-optimized TALE, split-halves of a deaminase and, optionally, at least one UGI. The systems of the present disclosure include a serial of DNA vectors and protocols to use them. The systems are used to change specific nucleotides (e.g., create premature stop codon of organelle genes, correct the deleterious DNA sequences, incorporate superior variants of DNA elements, etc.) in the genomes of organelles in plants, wherein the CRISPR-based genome editing is limited.

### EXAMPLES

Modular assembly of TALe repeats. The method for modular assembly of TALe repeats in 51 plasmids was performed as described in Li et al., 2011. Briefly, 3 arrays of 8 repeats in total of 23 TALE repeats were individually assembled. For each array of 8 repeats, one repeat-containing plasmid from each of the 8 repeat sets was chosen based on sequence (e.g., A, T, G and C) and the order (1 to 8) of DNA target by a particular cpDdCBE. The 8 TALE repeats were further assembled using the Golden Gate ligation method using the restriction enzyme BsmBI and T4 DNA ligase. The correct insertion of 8 repeats was confirmed by digestion of plasmid DNA with restriction enzymes PstI and XbaI (first octamers), PstI and XhoI (second octamers) and XhoI and BsrGI (third octamers) and electrophoresis for right lengths of 8 repeats. Finally, the putative clones were further confirmed for accuracy by Sanger sequencing the array using either oligonucleotides Seq-F or Seq-R. To assemble the 3 repeat arrays into the scaffold vectors of cpDdCBE-L and cpDdCBE-R, the first repeat array was digested with SphI and PstI, the second array was digested with PstI and BsrGI, and the third array was digested with BsrGI and AatII. The pKS/cpDdCBE-L and pKS/cpDdCBE-R each were digested with SphI and AatII. The vector and the three repeat arrays were ligated in one ligation reaction and confirmed by digestion with the restriction enzymes Acc65I and SacI.

Construction of Expression Plasmids. To make constructs that express paired TAL deaminases in plant cells (e.g., rice and maize), a system of two vectors was adapted. An intermediate vector, pENTR-OsUbi_p, and a destination vector pZmUbi_p-GW were used to clone the assembled TALCDA-R and TALCDA-L under the rice ubiquitin and maize ubiquitin promoters, respectively, at Acc65I and SacI. In brief, cpTALCDA-L coding region was cut out from pKS/cpTALCDA-L with Acc65I and SacI and purified from agarose gel; while vector pZmUbi_p-GW was digested with Acc65I and SacI and purified. The DNA fragment of cpTALCDA-L and pZmUbi_p-GW vector was ligated together, resulting in pZmUbi_p::T ALCDA-L-GW. Similarly, cloning of cpTALCDA-R lead to pENTR-OsUbip::TALCDA-R. Through Gateway reaction, the expression cassette of OsUbi_p::TALCDA-R was mobilized into pZmUbi_p::TALCDA-L-GW at the Gateway recipient site AttR1-AttR2, resulting in pZmUbi_p::TALCDA-L-OsUbi-p::TALCDA-R. The expression constructs contained hygromycin resistance for rice transformation selection, bialaphos resistance for maize transformation selection or green fluorescence protein genes for transient expression assay based on the destination vectors are used. Similarly, the chloroplast editing constructs were made for wheat and used for transformation of wheat (e.g., cultivar Fielder) using hygromycin resistance selection marker. For TAL deaminases derived from a single deaminase domain, the intermediate vector pKS/cpTAL-CDA was used to clone specific TAL DNA binding domain in. The resulting constructs were used to extract DNA fragment at Acc65I and SacI, and cloned into pZmUbi_p. The plasmids were transferred into Agrobacterium for rice transformation.

Transient Assay of cpTAL and mtTAL Deaminase in Rice, Maize and Wheat Protoplasts. The rice cultivar Kitaake, maize inbreds line B73, and wheat cultivar Fielder were used to produce seedlings of 7-10 days old for protoplast isolation by using a protocol described (Zhang et al., 2011). The protoplasts were infected with DNA constructs expressing three combinations of genes: 1.)35S::GFP alone; 2.) 35S::GFP+ZmUbi pro::cpPsaA3-L+OsUbi pro::cpPsaA3-R; 3) 35S::GFP+ZmUbi pro::cpPsaA4-L+OsUbi pro::cpPsaA4-R. The transfected protoplasts were kept at 28° C. under dark condition. The fluorescent protoplasts 36 hours post transfection were isolated and collected through fluorescence-activated cell sorting (FACS). About 10,000 fluorescent protoplasts from individual construct combinations were used for total DNA extraction.

Stable transgenic plants of rice, wheat, maize. The rice cultivar Kitaake, maize inbreds line B73, and wheat cultivar Fielder were used for transformation with respective TALE deaminase constructs by Agrobacterium- and/or particle bombardment-mediated gene delivery. Transgenic plants were selected with hygromycin (rice, wheat) and bialaphos (maize), and further genotyped for presence of transgenes and genotyped for presence of chloroplast or mitochondrial gene editing.

DNA Extraction and PCR amplification. The CTAB method was used to extract total DNA (nuclear, chloroplast and mitochondrial) as described (Murray et al., 1980). Gene and site-specific oligonucleotides (Table 1) were used to PCR-amplify the relevant regions from the chloroplast genes PsaA3. The PCR amplicons were initially subjected to Sanger sequencing to detect the potential DNA changes at the spacer regions of paired TAL deaminases.

Table 1 provides the primer names and sequences used in the Examples.

TABLE 1
Primers

| | | SEQ | |
|---|---|---|---|
| Primer | | ID | |
| Name | Sequence (5′ to 3′) | NO | Use |
| PsaA3-F1 | aagtatccgcctgggatcat | 10 | To detect base editing in |
| PsaA3-R1 | cagcacgtccttgtataatgc | 11 | PsaA in rice and maize |
| MiSsaA3-F | CTCTTTCCCTACACGACgctctt | 12 | Barcoded oligos to detect |
| | ccgatcTaagtatccgcctgggatcat | | base editing of PsaA in |
| MiSsaA3-R | CTGGAGTTCAGACGTGTGC | 13 | rice and maize |
| | TCTTCCGATCTcagcacgtccttgt | | |
| | ataatgc | | |
| Seq-F | TGGCCCGTGTCTCAAAATC | 14 | Oligos to sequence the |
| | TCTG | | TALE repeats assembled |
| Seq-R | ATCTTTTCTACGGGGTCTG | 15 | from modular units |
| | ACG | | |
| psbA-F3 | taccatgactgcaattttagag | 16 | To detect base editing in |
| psbA-F3 | CCGAATACACCAGCTACA | 17 | PsbA |
| | CCT | | |
| MiSsbA3- | CTCTTTCCCTACACGACgctcttc | 18 | Barcoded oligos to detect |
| F4 | cgatcTaccatgactgcaattttagag | | |
| MiSsbA3- | CTGGAGTTCAGACGTGTGC | 19 | base editing of PsbA |
| R4 | TCTTCCGATCTttgcggtcaataag | | |
| | gtaggg | | |
| ATP6-F | GCCATGTGATCGCTACTAAAG | 20 | To detect base editing in |
| ATP6-R | GCATTTGGCACTGACTTTCC | 21 | ATP6 |
| MiATP6-F | CTCTTTCCCTACACGACgctcttc | 22 | Barcoded oligos to detect |
| | cgatcTgccatgtgatcgctactaaag | | base editing of ATP6 |
| MiATP6-R | CTGGAGTTCAGACGTGTGC | 23 | |
| | TCTTCCGATCTGCTTGTCT | | |
| | CCTTCTCTTCAACG | | |

The compositions and methods disclosed herein are useful for changing specific nucleotides (e.g., create premature stop codon of organelle genes, correct the deleterious DNA sequences, incorporate superior variants of DNA elements, etc.) in the genomes of organelles in plants, wherein the application of CRISPR-based genome editing is limited.

1. A recombinant fusion protein comprising
a targeting peptide selected from the group consisting of a plant chloroplast targeting peptide and a plant mitochondrial targeting peptide,
a TALE array protein, and
a deaminase.

2. The recombinant fusion protein of claim 1, wherein the deaminase is a cytidine deaminase.

3. The recombinant fusion protein of claim 2, wherein the deaminase is a SCP1.201 deaminase.

4. The recombinant fusion protein of claim 3, wherein the SCP1.201 deaminase is selected from the group consisting of DddA, a SCPa deaminase, a SCPb deaminase, and a SCPc deaminase.

5. The recombinant fusion protein of claim 4, wherein the DddA is selected from the group consisting of an N-terminal fragment of DddA and a C-terminal fragment of Ddda.

6. The recombinant fusion protein of claim 1, further comprising at least one uracil glycosylase inhibitor.

7. The recombinant fusion protein of claim 6, wherein uracil glycosylase inhibitor is located at the N-terminus.

8. The recombinant fusion protein of claim 6, wherein the uracil glycosylase inhibitor is located at the C-terminus.

9. The recombinant fusion protein of claim 6, comprising two uracil glycosylase inhibitors.

10. A nucleic acid encoding the recombinant fusion protein of claim 1.

11. A vector comprising the nucleic acid of claim 10.

12. A plant, a plant cell, a plant tissue comprising the nucleic acid of claim 10.

13. A plant, a plant cell, a plant tissue comprising the recombinant fusion protein of claim 1.

14. A method of editing a plant chloroplast nucleic acid, the method comprising:
providing a recombinant fusion protein comprising a plant chloroplast targeting peptide, a TALE array protein, and a deaminase, wherein the recombinant fusion protein localizes to a plant chloroplast and forms a complex with a target chloroplast double-stranded nucleic acid to catalyze a C·to T·A conversion in the target chloroplast double-stranded nucleic acid.

15. The method of claim 14, wherein the recombinant fusion protein further comprises at least one UGI.

16.-17. (canceled)

18. A method of editing a plant mitochondria nucleic acid, the method comprising:
providing a recombinant fusion protein comprising a plant mitochondria targeting peptide, a TALE array protein, and a deaminase, wherein the recombinant fusion protein localizes to a plant mitochondria and forms a complex with a target mitochondria double-stranded nucleic acid to catalyze a C·G to T·A conversion in the target mitochondria double-stranded nucleic acid.

19. The method of claim 18, wherein the recombinant fusion protein further comprises at least one UGI.

20.-21. (canceled)


SEQUENCE LISTING (43 sequences)

SEQ ID NO: 1 - DNA, 4136 bp, Artificial Sequence
  Synthetic
aaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccagatctgccaccatggcgcccaccgtgatgatggcctcgtcggcaaccgccgtcgccccgttccagggtctcaagtccgccgccagcctccccgtcgcccgccgcagcaccaggagcctcggcaacgtcagcaacggcggaaggatccggtgtatgcagggatccgatctccgcacactcggatacagccagcagcagcaggagaagatcaaaccaaaggtgaggtctacagtggcccagcaccacgaggcactcgtgggacacgggtttacacacgcccacatcgttgctctcagccagcacccagcagccctcgggaccgttgctgtcacatatcaggacatcatcagggccttgccagaggccacacacgaagacatcgttggagtcggaaaacagtggtccggagcacgcgctctcgaagccttgctcacagaggccggggagttgaggggaccaccactccagttggacacaggacagcttctcaagattgcaaaaaggggaggagtgaccgcagtggaggcagtgcatgcggcggcgacgtcctgccctcgatgcagtgaaaaagggattgccacacgccccagaattgatcaggaggatcaatcgccgcattcctgaacgcacatcccaccgcgttcctagcggtggttcctacgccctcgggccgtatcagatttccgcccctcagttgccagcatacaacgggcaaactgtgggaacattttactatgtcaatgatgcagggggcctcgaatctaaggtcttttcaagcggtggtccaacaccctaccctaactacgctaatgctgggcatgtggagggccagtcggcgttgttcatgagggataatggaatatccgaaggtcttgtttttcacaacaatccggagggcacatgcggtttctgtgttaatatgacagaaacgctgttgccagagaacgccaagatgacagtcgtgccgcccgaggggtcgggtggttcaacaaacctgtcggatattatagaaaaggaaacaggtaagcagctcgtcattcaagagtcgatcctcatgctccccgaagaggttgaggaggttatcggtaacaagccagaatccgatatacttgtccataccgcctatgacgagagcacggatgagaacgttatgcttctgacatccgatgcccctgaatacaagccttgggcattggtgatacaagattcaaacggtgaaaataagataaaaatgctttagactagttaatctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 2 - DNA, 3904 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccagatctgccaccatggcgcccaccgtgatgatggcctcgtcggcaaccgccgtcgccccgttccagggtctcaagtccgccgccagcctccccgtcgcccgccgcagcaccaggagcctcggcaacgtcagcaacggcggaaggatccggtgtatgcagggatccgatctccgcacactcggatacagccagcagcagcaggagaagatcaaaccaaaggtgaggtctacagtggcccagcaccacgaggcactcgtgggacacgggtttacacacgcccacatcgttgctctcagccagcacccagcagccctcgggaccgttgctgtcacatatcaggacatcatcagggccttgccagaggccacacacgaagacatcgttggagtcggaaaacagtggtccggagcacgcgctctcgaagccttgctcacagaggccggggagttgaggggaccaccactccagttggacacaggacagcttctcaagattgcaaaaaggggaggagtgaccgcagtggaggcagtgcatgcggcggcgacgtcctgccctcgatgcagtgaaaaagggattgccacacgccccagaattgatcaggaggatcaatcgccgcattcctgaacgcacatcccaccgcgttcctagcggtgctattcctgtgaagcgcggcgcgactggggaaacgaaggtgtttaccggcaattccaactcccccaagtctcccacgaaggggggttgctccggtggctcgaccaatctctctgacatcatcgagaaggagactggcaaacaactggttattcaagaatccatattgatgctccccgaagaggtcgaagaggtcatagggaacaagcctgaatcagacatactggttcacaccgcctatgatgaaagcactgacgaaaatgtgatgctgctcacatcggacgcccccgagtataaaccctgggcgcttgtcatacaagactcgaacggagagaacaagataaagatgctctagactagttaatctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 3 - DNA, 4174 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccagatctgccaccatggcgtcacgcagacttctggcttcattgctccggcagtccgctcagagaggcgggggccttatttctcggtcactcggaaattccatcccaaagtctgcgtcaagggcgagttcgcgcgcttcaccaaaaggcttcttgctcaatagagcggtgcaatatgcaacgagtgcagcggctcaaggatccgatctccgcacactcggatacagccagcagcagcaggagaagatcaaaccaaaggtgaggtctacagtggcccagcaccacgaggcactcgtgggacacgggtttacacacgcccacatcgttgctctcagccagcacccagcagccctcgggaccgttgctgtcacatatcaggacatcatcagggccttgccagaggccacacacgaagacatcgttggagtcggaaaacagtggtccggagcacgcgctctcgaagccttgctcacagaggccggggagttgaggggaccaccactccagttggacacaggacagcttctcaagattgcaaaaaggggaggagtgaccgcagtggaggcagtgcatgcggcggcgacgtcctgccctcgatgcagtgaaaaagggattgccacacgccccagaattgatcaggaggatcaatcgccgcattcctgaacgcacatcccaccgcgttcctagcggtggttcctacgccctcgggccgtatcagatttccgcccctcagttgccagcatacaacgggcaaactgtgggaacattttactatgtcaatgatgcagggggcctcgaatctaaggtcttttcaagcggtggtccaacaccctaccctaactacgctaatgctgggcatgtggagggccagtcggcgttgttcatgagggataatggaatatccgaaggtcttgtttttcacaacaatccggagggcacatgcggtttctgtgttaatatgacagaaacgctgttgccagagaacgccaagatgacagtcgtgccgcccgaggggtcgggtggttcaacaaacctgtcggatattatagaaaaggaaacaggtaagcagctcgtcattcaagagtcgatcctcatgctccccgaagaggttgaggaggttatcggtaacaagccagaatccgatatacttgtccataccgcctatgacgagagcacggatgagaacgttatgcttctgacatccgatgcccctgaatacaagccttgggcattggtgatacaagattcaaacggtgaaaataagataaaaatgctttagactagttaatctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 4 - DNA, 3938 bp, Artificial Sequence
  Synthetic
aaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccagatctgccaccatggcgtcacgcagacttctggcttcattgctccggcagtccgctcagagaggcgggggccttatttctcggtcactcggaaattccatcccaaagtctgcgtcaagggcgagttcgcgcgcttcaccaaaaggcttcttgctcaatagagcggtgcaatatgcaacgagtgcagcggctcaaggatccgatctccgcacactcggatacagccagcagcagcaggagaagatcaaaccaaaggtgaggtctacagtggcccagcaccacgaggcactcgtgggacacgggtttacacacgcccacatcgttgctctcagccagcacccagcagccctcgggaccgttgctgtcacatatcaggacatcatcagggccttgccagaggccacacacgaagacatcgttggagtcggaaaacagtggtccggagcacgcgctctcgaagccttgctcacagaggccggggagttgaggggaccaccactccagttggacacaggacagcttctcaagattgcaaaaaggggaggagtgaccgcagtggaggcagtgcatgcggcggcgacgtcctgccctcgatgcagtgaaaaagggattgccacacgccccagaattgatcaggaggatcaatcgccgcattcctgaacgcacatcccaccgcgttcctagcggtgctattcctgtgaagcgcggcgcgactggggaaacgaaggtgtttaccggcaattccaactcccccaagtctcccacgaaggggggttgctccggtggctcgaccaatctctctgacatcatcgagaaggagactggcaaacaactggttattcaagaatccatattgatgctccccgaagaggtcgaagaggtcatagggaacaagcctgaatcagacatactggttcacaccgcctatgatgaaagcactgacgaaaatgtgatgctgctcacatcggacgcccccgagtataaaccctgggcgcttgtcatacaagactcgaacggagagaacaagataaagatgctctagactagttaatctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 5 - DNA, 4291 bp, Artificial Sequence
  Synthetic
ctttcctgcgttatcccctgattctgtggataaccgtattaccgctagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggctccaccatgggaaccaattcagtcgacaaattcgggtcaaggcggaagccagcgcgccaccccacgtcagcaaatacggaggcgcggggttgacggcgtcacccggtcctaacggcgaccaacaaaccagccagaagaaattacagtaaaaaaaaagtaaattgcactttgatccaccttttattacctaagtctcaatttggatcacccttaaacctatcttttcaatttgggccgggttgtggtttggactaccatgaacaacttttcgtcatgtctaacttccctttcagcaaacatatgaaccatatatagaggagatcggccgtatactagagctgatgtgtttaaggtcgttgattgcacgagaaaaaaaaatccaaatcgcaacaatagcaaatttatctggttcaaagtgaaaagatatgtttaaaggtagtccaaagtaaaacttatagataataaaatgtggtccaaagcgtaattcactcaaaaaaaatcaacgagacgtgtaccaaacggagacaaacggcatcttctcgaaatttcccaaccgctcgctcgcccgcctcgtcttcccggaaaccgcggtggtttcagcgtggcggattctccaagcagacggagacgtcacggcacgggactcctcccaccacccaaccgccataaataccagccccctcatctcctctcctcgcatcagctccacccccgaaaaatttctccccaatctcgcgaggctctcgtcgtcgaatcgaatcctctcgcgtcctcaaggtacgctgcttctcctctcctcgcttcgtttcgattcgatttcggacgggtgaggttgttttgttgctagatccgattggtggttagggttgtcgatgtgattatcgtgagatgtttaggggttgtagatctgatggttgtgatttgggcacggttggttcgataggtggaatcgtggttaggttttgggattggatgttggttctgatgattggggggaatttttacggttagatgaattgttggatgattcgattggggaaatcggtgtagatctgttggggaattgtggaactagtcatgcctgagtgattggtgcgatttgtagcgtgttccatcttgtaggccttgttgcgagcatgttcagatctactgttccgctcttgattgagttattggtgccatgggttggtgcaaacacaggctttaatatgttatatctgttttgtgtttgatgtagatctgtagggtagttcttcttagacatggttcaattatgtagcttgtgcgtttcgatttgatttcatatgttcacagattagataatgatgaactcttttaattaattgtcaatggtaaataggaagtcttgtcgctatatctgtcataatgatctcatgttactatctgccagtaatttatgctaagaactatattagaatatcatgttacaatctgtagtaatatcatgttacaatctgtagttcatctatataatctattgtggtaatttctttttactatctgtgtgaagattattgccactagttcattctacttatttctgaagttcaggatacgtgtgctgttactacctatctgaatacatgtgtgatgtgcctgttactatctttttgaatacatgtatgttctgttggaatatgtttgctgtttgatccgttgttgtgtccttaatcttgtgctagttcttaccctatctgtttggtgattatttcttgcagatccgaattcggtacccggggatccgtcgacccatggaagcttctagactagtgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagtctagacccagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctggtgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 6 - DNA, 11719 bp, Artificial Sequence
  Synthetic
gaattcaagcttgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcactagtaagcttggatccggtaccgggaagcatgggcccctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccgtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcctgcaggcatgcaagcttcaccactttgtacaagaaagctgaacgagaaacgtaaaatgatataaatatcaatatattaaattagattttgcataaaaaacagactacataatgctgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatcgatcctaactcaaaatccacacattatacgagccggaagcataaagtgtaaagcctggggtgcctaatgcggccgccaatatgactggatatgttgtgttttacagtattatgtagtctgttttttatgcaaaatctaatttaatatattgatatttatatcattttacgtttctcgttcagcttttttgtacaaacttgtgatccccgtctctagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagactcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattac

SEQ ID NO: 7 - DNA, 147 bp, Artificial Sequence
  Synthetic
atggcgcccaccgtgatgatggcctcgtcggcaaccgccgtcgccccgttccagggtctcaagtccgccgccagcctccccgtcgcccgccgcagcaccaggagcctcggcaacgtcagcaacggcggaaggatccggtgtatgcag

SEQ ID NO: 8 - DNA, 180 bp, Artificial Sequence
  Synthetic
atggcgtcacgcagacttctggcttcattgctccggcagtccgctcagagaggcgggggccttatttctcggtcactcggaaattccatcccaaagtctgcgtcaagggcgagttcgcgcgcttcaccaaaaggcttcttgctcaatagagcggtgcaatatgcaacgagtgcagcggct

SEQ ID NO: 9 - protein, 84 aa, Artificial Sequence
  Synthetic
MTNLSDIIEKETGKQLVIQESILMLPEEVEEVIGNKPESDILVHTAYDESTDENVMLLTSDAPEYKPWALVIQDSNGENKIKML

SEQ ID NO: 10 - DNA, 20 bp, Artificial Sequence
  Synthetic
aagtatccgcctgggatcat

SEQ ID NO: 11 - DNA, 21 bp, Artificial Sequence
  Synthetic
cagcacgtccttgtataatgc

SEQ ID NO: 12 - DNA, 50 bp, Artificial Sequence
  Synthetic
ctctttccctacacgacgctcttccgatctaagtatccgcctgggatcat

SEQ ID NO: 13 - DNA, 51 bp, Artificial Sequence
  Synthetic
ctggagttcagacgtgtgctcttccgatctcagcacgtccttgtataatgc

SEQ ID NO: 14 - DNA, 23 bp, Artificial Sequence
  Synthetic
tggcccgtgtctcaaaatctctg

SEQ ID NO: 15 - DNA, 22 bp, Artificial Sequence
  Synthetic
atcttttctacggggtctgacg

SEQ ID NO: 16 - DNA, 22 bp, Artificial Sequence
  Synthetic
taccatgactgcaattttagag

SEQ ID NO: 17 - DNA, 21 bp, Artificial Sequence
  Synthetic
ccgaatacaccagctacacct

SEQ ID NO: 18 - DNA, 51 bp, Artificial Sequence
  Synthetic
ctctttccctacacgacgctcttccgatctaccatgactgcaattttagag

SEQ ID NO: 19 - DNA, 50 bp, Artificial Sequence
  Synthetic
ctggagttcagacgtgtgctcttccgatctttgcggtcaataaggtaggg

SEQ ID NO: 20 - DNA, 21 bp, Artificial Sequence
  Synthetic
gccatgtgatcgctactaaag

SEQ ID NO: 21 - DNA, 20 bp, Artificial Sequence
  Synthetic
gcatttggcactgactttcc

SEQ ID NO: 22 - DNA, 51 bp, Artificial Sequence
  Synthetic
ctctttccctacacgacgctcttccgatctgccatgtgatcgctactaaag

SEQ ID NO: 23 - DNA, 52 bp, Artificial Sequence
  Synthetic
ctggagttcagacgtgtgctcttccgatctgcttgtctccttctcttcaacg

SEQ ID NO: 24 - protein, 126 aa, Actinokineospora iranica
LPPDIIRSDNRPRGTPQPKTHGRWIDPDGNTHAEVSGWDDKYRAAVNWFAAQPGTKVPSTVADVEIKLAVHMRQNAIRYATRAINHVPCKGDFGCDTLVPHILPEGYILVVHGAGGFRKTYHGRAK

SEQ ID NO: 25 - protein, 85 aa, Actinokineospora iranica
LRDLGFAPDRAGRYPAASHAEVKLACWMRETGVKHVVAVINNSKGVCADEDQTCDAFTSALPPRGYLLEVWSPGKAKPTKLPGGA

SEQ ID NO: 26 - protein, 132 aa, Propionibacterium acidifaciens
PTTPQGTSSDTIAEMLNSASQPGRTAGVLDIDGELTPLTSGRPSLPNYIASGHVEGQAAMIMRQQQVQSATVYHDNPNGTCGYCYSQLPTLLPEGAALDVVPPAGTVPPSNRWHNGGPSFIGNSSEPKPWPR

SEQ ID NO: 27 - protein, 60 aa, Nicotiana plubaginifolia
MASRRLLASLLRQSAQRGGGLISRSLGNSIPKSASRASSRASPKGFLLNRAVQYATSAAA

SEQ ID NO: 28 - DNA, 64 bp, Artificial Sequence
  Synthetic
tacaagcctgtggggtcgcttctgcaactggataactagcaccgaaaaccgtctttacatcgga

SEQ ID NO: 29 - DNA, 64 bp, Artificial Sequence
  Synthetic
  misc_feature (30)..(30): N is not G
  misc_feature (31)..(31): N is not G
tacaagcctgtggggtcgcttctgcaactnnataactagcaccgaaaaccgtctttacatcgga

SEQ ID NO: 30 - DNA, 64 bp, Artificial Sequence
  Synthetic
  misc_feature (30)..(30): N is not G
  misc_feature (31)..(31): N is not G
  misc_feature (36)..(36): N is not C
tacaagcctgtggggtcgcttctgcaactnnataantagcaccgaaaaccgtctttacatcgga

SEQ ID NO: 31 - DNA, 64 bp, Artificial Sequence
  Synthetic
tttcggtagtcattttccatttcagttggaaaatgcagtcggatgtttggggtactataagtga

SEQ ID NO: 32 - DNA, 64 bp, Artificial Sequence
  Synthetic
tttcggtagtcattttccatttcagttaaaaaatgcagtcggatgtttggggtactataagtga

SEQ ID NO: 33 - DNA, 64 bp, Artificial Sequence
  Synthetic
tttcggtagtcattttccatttcagttgaaaaatgcagtcggatgtttggggtactataagtga

SEQ ID NO: 34 - DNA, 67 bp, Artificial Sequence
  Synthetic
tcgatcacaatcatgtggtaataatgggtttgaatcagagagactcgatctggaaactcctcaatgt

SEQ ID NO: 35 - DNA, 65 bp, Artificial Sequence
  Synthetic
cgatcacaatcatgtggtaataatgggtttgaatcagagagactcgatctggaaactcctcaatg

SEQ ID NO: 36 - DNA, 65 bp, Artificial Sequence
  Synthetic
cgatcacaatcatgtggtaataatgggtttaaattagagagactcgatctggaaactcctcaatg

SEQ ID NO: 37 - DNA, 66 bp, Artificial Sequence
  Synthetic
tggcagaacggaatagagttgttagatggggctgaatggaggaacggcgatatagttatccctgga

SEQ ID NO: 38 - DNA, 68 bp, Artificial Sequence
  Synthetic
actggcagaacggaatagagttgttagatggggctgaatggaggaacggcgatatagttatccctgga

SEQ ID NO: 39 - DNA, 68 bp, Artificial Sequence
  Synthetic
  misc_feature (30)..(30): N is not G
actggcagaacggaatagagttgttagatnaagctaaatggaggaacggcgatatagttatccctgga

SEQ ID NO: 40 - DNA, 68 bp, Artificial Sequence
  Synthetic
  misc_feature (31)..(31): N is not G
actggcagaacggaatagagttgttagatgnggctgaatggaggaacggcgatatagttatccctgga

SEQ ID NO: 41 - DNA, 68 bp, Artificial Sequence
  Synthetic
  misc_feature (31)..(31): N is not G
  misc_feature (32)..(32): N is not G
actggcagaacggaatagagttgttagatgnngctgaatggaggaacggcgatatagttatccctgga

SEQ ID NO: 42 - DNA, 64 bp, Artificial Sequence
  Synthetic
tacaagcctgtggggtcgcttctgcaactggataactagcaccgaaaaccgtctttacattgga

SEQ ID NO: 43 - DNA, 64 bp, Artificial Sequence
  Synthetic
tacaagcctgtggggtcgcttctgcaactaaataactagcaccgaaaaccgtctttacattgga